(19) United States
(12) Patent Application Publication
(10) Pub. No.: US 2006/0242788 A1
(43) Pub. Date: Nov. 2, 2006

(54) Mouth guard cleansing and sanitizing system

(76) Inventor: Susan M. Day, Leonardtown, MD (US)

Correspondence Address: JAMES RAY & ASSOCIATES, 2640 PITCAIRN ROAD, MONROEVILLE, PA, 15146, US

(21) Appl. No.: 11/410,712

(22) Filed: Apr. 25, 2006

Related U.S. Application Data
(60) Provisional application No. 60/675,674, filed on Apr. 28, 2005.

(51) Int. Cl. A47L 9/04

(52) U.S. Cl. 15/363

(57) ABSTRACT

A portable, reusable cleansing system for mouth guards. The system comprises a case member for holding such mouth guard in one of an upper portion and a bottom portion thereof when such mouth guard is not in use. Such case member has a predetermined configuration, a predetermined size and a predetermined shape. There is a cleansing and sanitizing means that is disposed in an opposed one of such upper portion and such bottom portion of the case member for cleansing and sanitizing such mouth guard when such mouth guard is placed in the case member. An activating means is disposed one of on and in the case member for activating such cleansing and sanitizing means when such unsanitized mouth guard is placed in the case member. A power source is disposed in one of the upper portion and the bottom portion of the case member and externally for providing power to such cleansing and sanitizing means.

### CROSS REFERENCE TO RELATED APPLICATIONS

This application is closely related to and claims benefit from U.S. Provisional Application Ser. No. 60/675,674 filed Apr. 28, 2005.

### FIELD OF THE INVENTION

The present invention relates, in general, to a mouth guard, and, more particularly, the present invention relates to a mouth guard sanitizing system for cleaning and sanitizing mouth guards after use so the mouth guard will be clean and sanitary when it is used next.

### BACKGROUND OF THE INVENTION

Athletes tend to live out of their gear bag. Many sports today require that the players wear protective mouth guards. Currently, most mouth guards tend to be tossed into the gear bag after being used and the mouth guard remains there until it is used again. Occasionally, the athlete may rinse out the mouth guard with water before tossing it into the gear bag. This is a situation that can lead to a breeding ground for a variety of germs.

Today's on the go athlete simply does not have time or the inclination to properly disinfect his or her mouth guard on a continual basis.

Thus, it would be advantageous if there were a means for the athlete to clean and disinfect the mouth guard without involving too much of the athlete's time.

### SUMMARY OF THE INVENTION

In a first aspect the present invention provides a portable, reusable cleansing system for mouth guards. The portable, reusable system comprises a case member for holding such mouth guard in one of an upper portion and a bottom portion thereof when such mouth guard is not in use. Such case member has a predetermined configuration, a predetermined size and a predetermined shape. There is a cleansing and sanitizing means that is disposed in an opposed one of such upper portion and such bottom portion of the case member for cleansing and sanitizing such mouth guard when such mouth guard is placed in the case member. An activating means is disposed one of on and in the case member for activating such cleansing and sanitizing means when such unsanitized mouth guard is placed in the case member. A power source is disposed in one of the upper portion and the bottom portion of the case member and externally for providing power to such cleansing and sanitizing means.

### OBJECTS OF THE INVENTION

It is, therefore, one of the primary objects of the present invention to provide a system for cleaning and sanitizing mouth guards that is portable.

Another object of the present invention is to provide a system for cleaning and sanitizing mouth guards that is reusable.

Still another object of the present invention is to provide a system for cleaning and sanitizing mouth guards that is convenient and easy to use.

Yet another object of the present invention is to provide a system for cleaning and sanitizing mouth guards that will also store such mouth guard until the next use.

These and various other objects and advantages of this invention will become apparent after a full reading of the following detailed description, particularly, when read in conjunction with the attached drawings as described below and the appended claims.

### BRIEF DESCRIPTION OF THE DRAWINGS

FIG. 1 is a perspective view of the portable, reusable cleaning system according to an embodiment of the invention.

FIG. 2 is a perspective view of the portable, reusable cleaning system seen in FIG. 1 from a different angle.

FIG. 3 is a perspective view of the portable, reusable cleaning system seen in FIG. 1 with the lid closed.

FIG. 4 is a perspective view of the bottom portion of the portable, reusable cleaning system seen in FIG. 1 showing he power means.

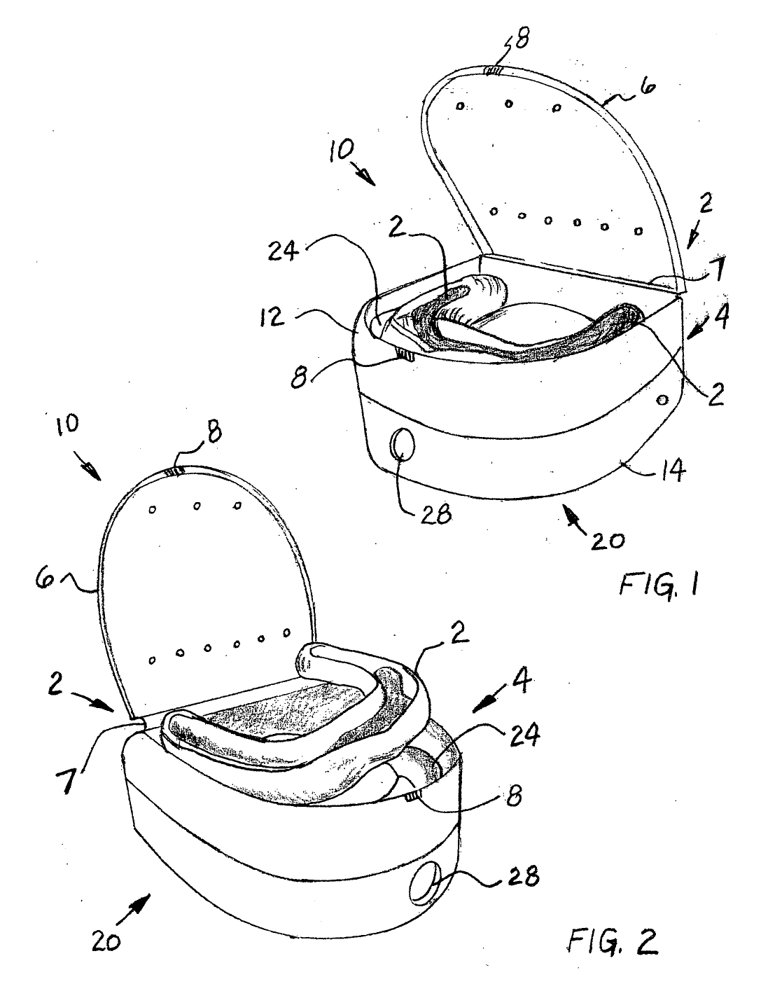

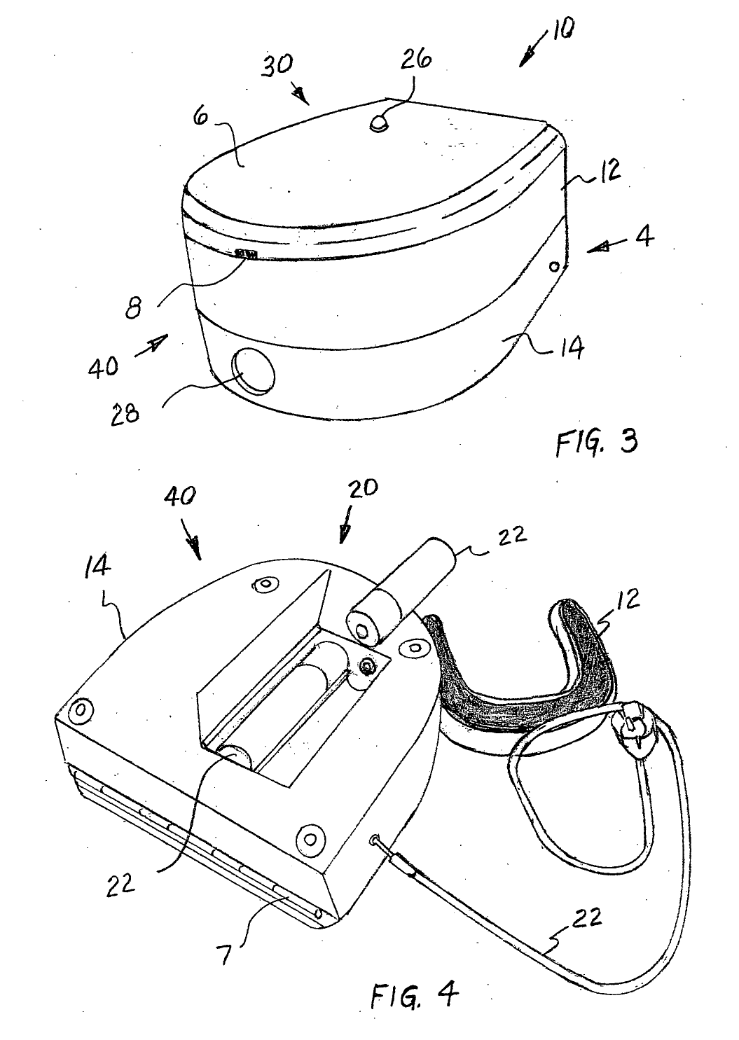

### DESCRIPTION OF THE PRESENTLY PREFERRED AND ALTERNATE EMBODIMENTS OF THE INVENTION

Prior to proceeding with the more detailed description of the present invention it should be noted that, for the sake of clarity, identical components which have identical functions have been designated by identical reference numerals throughout the several views illustrated in the drawings.

The present invention provides a portable, reusable cleansing system, generally designated 10, for mouth guards 2. Although all of the discussion is in relation to sports mouth guards, it should be noted that the present invention is equally applicable to other applications besides sports mouth guards. It is within the scope of the invention that such apparatus can also be used to sanitize dentures, removable retainers and bite guards as well as mouth guards.

The portable, reusable system 10 comprises a case member 4 for holding such mouth guard 2 in one of an upper portion 12 and a bottom portion 14 thereof when such mouth guard 2 is not in use. Such case member 4 has a predetermined configuration, a predetermined size and a predetermined shape. There is a cleansing and sanitizing means, generally designated 20, that is disposed in an opposed one of such upper portion 12 and such bottom portion 14 of the case member 4 for cleansing and sanitizing such mouth guard 2 when such mouth guard 2 is placed in the case member 4. An activating means, generally designated 30, is disposed one of on and in the case member 4 for activating such cleansing and sanitizing means 20 when such unsanitized mouth guard 2 is placed in the case member 4. A power source 22 is disposed in one of the upper portion 12 and the bottom portion 14 of the case member 4 and externally for providing power to such cleansing and sanitizing means 20.

It is presently preferred that such power source 22 is disposed in the bottom portion 14 of said case member 4 and that such upper portion 12 of the case member 4 is used for holding such mouth guard 2.

It is also preferred that such cleansing and sanitizing means 20 includes an ultraviolet light and that such ultraviolet light also is disposed in such bottom portion 14 of such case member 4. As seen in FIG. 4 such power source 22 can be one of an internal battery 22 or an exterior electrical means 22 such as connection to a 110 volt outlet. It is presently preferred that such power source 12 is an internal battery 22 which makes it easier for the athlete to use the cleaning and sanitizing means without the need of connecting to an outside power source. The case member 4 includes a tray member 24 which is used to separate the upper portion 12 and the bottom portion 14 of the case member 4 and hold the mouth guard 2. Thus, basically the mouth guard 2 is placed on the top of tray member 24 while the battery 22 and the cleansing and sanitizing means 20 is placed in the bottom portion 14 of the case member 4 below the tray member 24. Thus, it is important that the tray member 24 permit ultraviolet light to pass through such tray member 24 so as to clean and sanitize such mouth guard 2 that is placed on the tray member 24.

Such activation means 30 of the portable, reusable cleansing system 10 includes a button 26. Such button 26 is electrically connected with the power source (battery) 22 and the cleansing and sanitizing means 20 (ultraviolet light 20). Such system 10 also includes a timing means, generally designated 40 and an exterior lighted indicator 28 that is lit during a period of time that the cleansing and sanitizing operation 20 is in progress. Such timing means 40 is set for a predetermined time, substantially 10 minutes, which is the recommended time to sanitize such mouth guard 2 with the ultra violet light. Thus, when the button 26 is pushed to activate the cleansing and sanitizing means 20 such exterior lighted indicator 28 glows to show that the system is in operation. The ultra violet light will stay on for the predetermined period of time, approximately 10 minutes, set by the timing means 40, and the exterior light 28 will also stay on for that period of time. After the predetermined time the ultra violet light and the exterior light 28 will both be turned off and the mouth guard 2 will be cleansed and sanitized.

Such case member 4 also includes a lid member 6 which has a hinged back 7 and a fastener 8 that engages the case member 4 for insuring that lid member 8 stays closed when not in use. It is presently preferred that such case member which includes the upper portion, the bottom portion and the tray member were all made of plastic.

While a presently preferred embodiment and alternate embodiments of the present invention has been described in detail above, it should be understood that various other adaptations and/or modifications of the invention can be made by those persons who are particularly skilled in the art without departing from either the spirit of the invention or the scope of the appended claims.

I claim:

1. A portable, reusable cleansing system for mouth guards, said portable, reusable system comprising:
(a) a case member for holding such mouth guard in one of an upper portion and a bottom portion thereof when such mouth guard is not in use, said case member having a predetermined configuration, a predetermined size and a predetermined shape;
(b) a cleansing and sanitizing means disposed in a an opposed one of said upper portion and said bottom portion of said case member for cleansing and sanitizing such mouth guard when such mouth guard is placed in said case member;
(c) an activating means disposed one of on and in said case member for activating said cleansing and sanitizing means when such unsanitized mouth guard is placed in said case member; and
(b) a power source disposed in one of said upper portion and said bottom portion of said case member and externally for providing power to said cleansing and sanitizing means.

2. The portable, reusable cleansing system, according to claim 1, wherein said power source is disposed in said bottom portion of said case member.

3. The portable, reusable cleansing system, according to claim 2, wherein said upper portion of said case member is used for holding such mouth guard.

4. The portable, reusable cleansing system, according to claim 2, wherein said cleansing and sanitizing means is disposed in said bottom portion of said case member.

5. The portable, reusable cleansing system, according to claim 1, wherein said cleansing and sanitizing means includes an ultraviolet light.

6. The portable, reusable cleansing system, according to claim 1, wherein said power source is one of an internal battery and an electrical means.

7. The portable, reusable cleansing system, according to claim 6, wherein said power source is an internal battery.

8. The portable, reusable cleansing system, according to claim 1, wherein said upper portion and said bottom portion of said case member are separated by a tray member.

9. The portable, reusable cleansing system, according to claim 8, wherein said tray member permits ultraviolet light to pass through said tray member so as to clean and sanitize such mouth guard placed on said tray member.

10. The portable, reusable cleansing system, according to claim 1, wherein activation means includes a button.

11. The portable, reusable cleansing system, according to claim 8, wherein said button is electrically connected with said power source and said cleansing and sanitizing means.

12. The portable, reusable cleansing system, according to claim 1, wherein said case further includes an exterior lighted indicator that is lit during a period of time said cleansing and sanitizing operation is in progress.

13. The portable, reusable cleansing system, according to claim 1, wherein said portable, reusable cleansing system further includes a timing means.

14. The portable, reusable cleansing system, according to claim 13, wherein said timing means is set for a predetermined period of time for said power source to provide power to said cleansing and sanitizing means.

15. The portable, reusable cleansing system, according to claim 14, wherein said predetermined period of time is substantially ten minutes.

16. The portable, reusable cleansing system, according to claim 3, wherein said case member further includes a lid member.

17. The portable, reusable cleansing system, according to claim 16, wherein said lid member has a hinged back.

18. The portable, reusable cleansing system, according to claim 16, wherein said lid member further includes a fastener for engagement with said case member for insuring that said lid member stays closed when not in use.

19. The portable, reusable cleansing system, according to claim 1, wherein said case member, including said upper portion said bottom portion and said tray member are made of plastic.
